(19) United States
(12) Plant Patent Application Publication
(10) Pub. No.: US 2014/0373230 P1
(43) Pub. Date: Dec. 18, 2014

(54) Variety denomination: 'Evipo026'

(50) ;

(71) Applicants: Mogens Nyegaard Olesen, Fredensborg (DK); Raymond Evison, St. Sampsons (GB)

(72) Inventors: Mogens Nyegaard Olesen, Fredensborg (DK); Raymond Evison, St. Sampsons (GB)

(21) Appl. No.: 13/986,920

(22) Filed: Jun. 14, 2013

(51) Int. Cl. A01H 5/00

(52) U.S. Cl. PLT/228

(57) ABSTRACT

A new Clematis plant with a compact growth habit, profuse, violet blue flowers, and continuous summer flowering. The variety successfully propagates from softwood cuttings and is suitable for cultivation commercial nursery culture. This new and distinct variety has shown to be uniform and stable in the resulting generations from asexual propagation from vegetative cuttings.

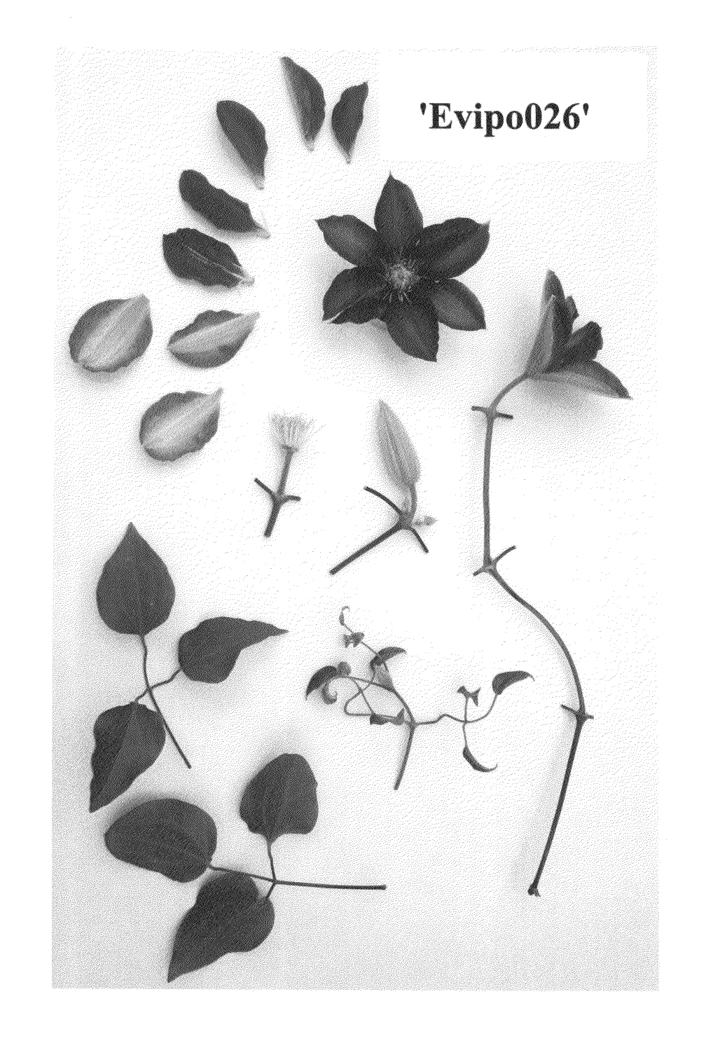

### BOTANICAL CLASSIFICATION

Genus: Clematis

Species: viticella

### VARIETY DENOMINATION

‘Evipo026’

### SUMMARY OF THE CLAIMED PLANT

The present invention constitutes a new and distinct variety of Clematis plant which originated from a controlled crossing between the female seed parent, an un-named seedling, and the male pollen parent, an un-named seedling

The two parents were crossed during the summer of 1992 and the resulting seeds were planted the following winter in a controlled environment in Guernsey, Channel Islands. The new variety named ‘Evipo026’ originated as a single seedling from the stated cross.

The new clematis plant may be distinguished from its female seed parent and male pollen parent primarily by flower color and growth habit.

The objective of the hybridization of this clematis plant was to create a new and distinct variety for nursery culture with unique qualities such as:
1. Uniform and abundant violet blue flowers;
2. Vigorous and compact growth, making the variety suitable for container culture; and
3. Improved disease resistance.

This combination of qualities was lacking in clematis plants that were in commercial cultivation and the qualities have been substantially achieved in the new variety.

‘Evipo026’ was selected by Mogens N. Olesen and Raymond J. Evison in their clematis development program in the Channel Islands, United Kingdom in 1993. Asexual reproduction of ‘Evipo026’ by means of vegetative cuttings and traditional layering was first performed by Mogens N. Olesen and Raymond J. Evison in the nursery during the summer of 1993. This initial and subsequent asexual propagations have demonstrated that the characteristics of ‘Evipo026’ are true to type and are transmitted from one generation to the next.

### BRIEF DESCRIPTION OF THE DRAWING

The accompanying color illustration show as true as is reasonably possible to obtain in color photographs of this type the typical characteristics of the buds, flowers, leaves, and stems, of ‘Evipo026’. Specifically illustrated in the drawing are open flowers, flower in parts, leaves, and stems.

### DETAILED DESCRIPTION OF THE VARIETY

The following is a detailed description of ‘Evipo026’, as observed in its growth throughout the flowering period in Yamhill County Oregon, USA. Observed plants were cultivated for a period of 18 months in 2 liter containers. Certain phenotypical characteristics of the variety may vary under different environmental, cultural, agronomic, seasonal, and climatic conditions. Color references are made using The Royal Horticultural Society (London, England) Colour Chart, 2001, except where common terms of color are used.

For a comparison, several physical characteristics of the clematis variety ‘Evipo037’ described and illustrated in U.S. Plant Pat. No. 18,104 are compared to ‘Evipo026’ in Chart 1.

CHART 1

| | ‘Evipo026’ | ‘Evipo037’ |
|---|---|---|
| Flower diameter | 90 mm | 130 to 150 mm |
| Upper tepal | Violet Blue Group | Violet-Blue |
| surface coloration | N92B at margins with | Group N89D |
| after opening: | mid-tepal coloration | |
| | of Violet Blue Group | |
| | 92A | |
| Tepal count | 8 to 9 | 6 to 7 |

Flower and flower bud:
   Blooming habit.—Recurrent. The natural flowering period is generally from late April to September.
   Flower bud.—Size: Normally 40 mm in length. Bud diameter is 15 mm. Bud form: Elliptic. Bud color: Yellow-Green Group 145 C. Texture: Pubescent.
   Peduncle.—Surface Texture: Smooth, somewhat striated, and pubescent. Length: On average 18 to 20 mm. Diameter: 3 mm. Color: Yellow-Green Group 145C. Strength: Somewhat weak.
   Flower arrangement.—Location on vine: New and old growth. Borne: Singly.
   Flower bloom.—Size: On average, flowers are 90 mm in diameter and 25 mm in depth. Profile: Flat to convex. Fragrance: None. Lasting Quality: Flowers normally remain 8 days on the plant.
   Tepals.—Tepal Color: The upper surface is Violet Blue Group N92B at margins with mid-tepal coloration of Violet Blue Group 92A. The lower surface is Violet Blue Group 93B at margins with a central bar, which is Yellow-Green Group 145C. Quantity: Normally 8 to 9 tepals. Size: 50 mm in length by 30 mm wide. Shape: Elliptic. The tepal apex is mucronate. The base is acute. Apex Recurvature: Slightly recurved. Tepal Cross section: Reflexed. Margins: Entire. Weak undulations. Persistence: Tepals drop off cleanly.
   Reproductive organs.—Arrangement: Compact. Pollen: Quantity: Moderate. Color: Yellow Group 4D. Anthers: Size: About 6 mm in length. Color: Yellow-Green Group 150D. Quantity: On average, 40. Filaments: Color: Green-White Group 157A. Length: About 5 mm. Pistils: Quantity: On average, 15. Stigmas: Green-White Group 157A. Styles: Color: Green-White Group 157A. Length: 11 mm.
   Seed head characteristics.—Not observed.
Plant:
   Plant form.—Climbing and spreading.
   Plant growth.—Moderately vigorous.
   Size.—Seasons growth attains 100 cm in height. Average spread is 75 cm.
   Hardiness.—Trials to date show the variety hardy in USDA Zones 5.
   Stems.—Color: Juvenile stems are Yellow-Green Group 146A. Mature stems are Greyed-Red Group 182A. Internodes: On average, 70 to 85 mm between nodes. Length: Normally 40 cm from the base of the plant to the flowering portion of the stem. Diameter: Normally 3 mm. Texture: Mature stems are pubescent, and ribbed.
   Plant foliage.—Leaf characteristics: Deciduous. Arrangement: Trifoliate. Leaf Size: Compound leaves are normally 130 mm (l)×110 mm (w). Leaflets are normally 56 mm (l)×40 mm (w). Abundance: On average 1 to 2 leaves per 10 cm of stem. Leaf Color: Juvenile upper Yellow-Green Group 146A. Juvenile lower Yellow-Green Group 146B. Mature upper Green Group 137B to Yellow-Green Group 146A. Mature lower surface is Yellow-Green Group 146B. Stipules: Absent. Petioles: Size: Normally 55 mm in length by 1.5 mm diameter. Texture: Smooth, and somewhat pubescent. Color: Greyed-Purple Group 187A. Petioloule: Size: Normally 20 to 30 mm in length by 1 mm diameter. Texture: Smooth, somewhat pubescent. Color: Greyed-Orange Group 176A. Leaflet Shape: Generally cordate. The apex is acute, base is rounded. Margin: Entire. Surface: The upper side is smooth. The lower side is smooth and pubescent. Thickness: Average. Glossiness: Somewhat glossy.
   Disease resistance.—Subject to any disease that normally attacks the species. However the variety is more tolerant to clematis wilt than some clematis.
   Cold hardiness.—The variety is tolerant to USDA Hardiness Zone 5.
   Heat tolerance.—The variety has been found to be suitable for climate conditions found in the American Horticulture Society heat zone 7.

We claim:

1. A new and distinct variety of clematis plant named ‘Evipo026’, substantially as described and illustrated, due to its abundant violet blue flowers with good keepability, attractive long lasting foliage and compact growth, year round flowering under glasshouse conditions, suitability for production from softwood cuttings in pots, durable flowers and foliage which make the variety suitable for distribution in the floral industry.
